(19) United States
(12) Patent Application Publication
(10) Pub. No.: US 2024/0082436 A1
(43) Pub. Date: Mar. 14, 2024

(54) SYNERGISTIC CANCER TREATMENT

(71) Applicant: ProLynx LLC, San Francisco, CA (US)

(72) Inventors: Daniel V. SANTI, San Francisco, CA (US); Shaun FONTAINE, Concord, CA (US)

(73) Assignee: ProLynx LLC, San Francisco, CA (US)

(21) Appl. No.: 18/220,742

(22) Filed: Jul. 11, 2023

Related U.S. Application Data
(63) Continuation of application No. 16/961,640, filed on Jul. 10, 2020, now Pat. No. 11,730,836.
(60) Provisional application No. 62/716,788, filed on Aug. 9, 2018.
(60) Provisional application No. 62/716,796, filed on Aug. 9, 2018.
(60) Provisional application No. 62/711,423, filed on Jul. 27, 2018.
(60) Provisional application No. 62/711,421, filed on Jul. 27, 2018.
(60) Provisional application No. 62/700,147, filed on Jul. 18, 2018.
(60) Provisional application No. 62/674,483, filed on May 21, 2018.
(60) Provisional application No. 62/617,095, filed on Jan. 12, 2018.

(51) Int. Cl. A61K 51/06; A61K 9/51; A61K 31/4745; A61K 47/60; A61P 35/00

(52) U.S. Cl. CPC A61K 51/065; A61K 9/51; A61K 31/4745; A61K 47/60; A61P 35/00; A61K 45/06

(57) ABSTRACT

Conjugates of topoisomerase I inhibitors linked to a macromolecule through a linkage that undergo beta elimination in situ in combination with one or more of an assessed defect in DNA damage response (DDR) in a subject bearing cancer, a cell cycle checkpoint inhibitor and/or a DDR inhibitor provides improved outcomes for cancer-bearing subjects.

### CROSS-REFERENCE TO RELATED APPLICATIONS

This application is a continuation of U.S. application Ser. No. 16/961,640, filed Jul. 10, 2020, which is a national stage application under 35 U.S.C. § 371 of International Application No. PCT/US2019/013314, filed internationally on Jan. 11, 2019, which claims priority from U.S. provisional application 62/617,095 filed Jan. 12, 2018, U.S. provisional application 62/674,483 filed May 21, 2018, U.S. provisional application 62/711,421 filed Jul. 27, 2018, U.S. provisional application 62/716,788 filed Aug. 9, 2018, U.S. provisional application 62/716,796 filed Aug. 9, 2018, U.S. provisional application 62/700,147 filed Jul. 18, 2018, and U.S. provisional application 62/711,423 filed Jul. 27, 2018, the disclosures of which are herein incorporated by reference in their entirety.

### TECHNICAL FIELD

The invention relates to use of topoisomerase I inhibitors linked to a macromolecule through a linkage that undergoes beta elimination for the treatment of cancer. More specifically, it concerns topoisomerase I inhibitor treatment of subjects having cancer where the pharmacokinetics are suitably controlled, said subjects having either a genetic defect in a DNA damage response (DDR) and/or said treatment involves administering a topoisomerase I inhibitor in combination with an inhibitor of DDR or in combination with a cell cycle checkpoint inhibitor. In some embodiments, the invention entails the exploitation of synthetic lethal interactions in cancer cells, where a defect in a gene necessary for DDR causes a second gene to become essential for cell survival.

### BACKGROUND ART

Topoisomerase I inhibitors are well known for treatment of various cancers, as they are inhibitors of the essential ligation step catalyzed by topoisomerase I to remedy single strand DNA damage that occurs due to relief of tension caused by supercoiling in DNA replication. (DNA replication requires topoisomerase I.) Topoisomerase I inhibitors include camptothecin and analogs thereof. Many of these compounds are approved and used in chemotherapy in the treatment of a wide variety of cancers.

In cancer cells with certain genetic defects the administration of a topoisomerase I inhibitor has been observed to have enhanced efficacy as compared to cancer cells without such defects. For example, one topoisomerase inhibitor, SN-38, has been administered as a conjugate with polyethylene glycol in BRCA1-deficient mice with mammary tumors and not only is the combination of BRCA1 deficiency with the inhibition by SN-38 of topoisomerase effective, but it also overcomes ABCG2 mediated resistance. See, for example, Zander, S. A. L. et al., PLOS One (2012) 7:e45248. In addition, various topoisomerase inhibitors have been administered in combination with additional anticancer agents that are DDR inhibitors and/or cell cycle checkpoint inhibitors. See, for example, Abal, M. et al., Oncol. Gene (2004) 23:1737-1744, Wainberg, Z. A. et al., Targ Oncol. (2017) 12:775-785; Verschraegen, C. F. et al., Cancer (2013) 5:418-429; and Gray, J. et al., Cancer Biol. and Ther. (2012) 13:614-622; Josse, R e. al, Cancer Res (2014) 74:6968-6978; Ma, C. X., et al, Breast Cancer Res Treat (2013) 137:483-492. In vitro studies have also shown that inhibiting expression of a protein important in DNA replication and repair, Werner Syndrome helicase (WRN) enhances the effect of irinotecan on cancer cells. See Futami, K., et al., Biol Pharm Bull (2007) 30:1958-1961. Combination of cell checkpoint inhibitors with various DNA damaging drugs has also been tested in clinical trials. (See, Visconti, R. et al., J. Exp. Clin. Cancer Res. (2016) 35:153.)

In addition, there is extensive knowledge of the landscape of DNA damage response deficiencies across various genes and genome locations (see Knijnenburg, T. A. et al., Cell Reports (2018) 23:239-254.

Coupling of topoisomerase I inhibitors, including SN-38, to macromolecules has been reported by Zhao, H. et al., Bioconjugate Chem. (2008) 19:849-859 and Koizumi, F. et al., Cancer Res. (2006) 66:10048-10056. A particular set of conjugates useful in the invention is disclosed by Santi, D. V. et al., J. Med. Chem. (2014) 57:2303-2314. An additional conjugate commonly denoted NKTR-102, which is a PEGylated irinotecan, is also known.

The present invention provides improved methods of treatment with topoisomerase I inhibitors in tumor subjects, which methods take advantage of either an inherent defect in DDR of a subject either associated with a germline mutation or other dysfunction in the cancer cells of the subject or combination treatment with additional agents that result in synthetic lethality.

### DISCLOSURE OF THE INVENTION

As evidenced by the literature cited above, it is known that topoisomerase I is essential for DNA replication, which is essential for cell growth and for replication. Inhibitors of topoisomerase I, such as irinotecan and its active metabolite, SN-38, have been used to treat cancer by inhibiting successful DNA replication.

There are also reports of attempts to combine topoisomerase I inhibitors with either a cell cycle checkpoint inhibitor (which neutralizes the mechanism by which cells determine that replication has or has not been successfully accomplished) or an additional inhibitor of DNA damage response (DDR). It is also known to administer topoisomerase I inhibitors to cancers already characterized as deficient in DDR.

Some such attempts have involved topoisomerase I inhibitors coupled to a solubilizing agent such as polyethylene glycol (PEG). However, the pharmacokinetics of the inhibitors thus far provided have not been appropriate to obtain a successful result and toxicity of these inhibitors has also been problematic.

The protocols of the invention are most importantly performed in human subjects, although the invention is also applicable to other mammalian subjects, including laboratory models for testing disease treatments. The protocols are also useful in livestock and companion animals.

It has now been found that by providing a topoisomerase I inhibitor with a linkage to a macromolecule that decouples through beta elimination, the pharmacokinetics can be adjusted to provide more effective and tolerable treatments in a subject having a defect in DDR, or in combination with inhibitors of a cell cycle checkpoint pathway and/or with an inhibitor of DDR. The conjugates of the invention can also be administered in doses that mitigate the synergistic toxicity of topoisomerase I inhibitors with such additional agents.

Thus, in a first aspect, the invention is directed to a method to treat cancer in a subject in need of such treatment, said subject having been identified as having one or more defects in DNA damage response (DDR). The method comprises administering to the subject an effective amount of a topoisomerase I inhibitor coupled to a macromolecule through a linker that provides decoupling through a beta elimination mechanism.

In a second aspect, the invention is directed to a method to treat cancer in a subject, which comprises administering to the subject an effective amount of a topoisomerase I inhibitor coupled to a macromolecule through a linker that provides decoupling through a beta elimination mechanism in combination with an effective amount of an additional inhibitor of DDR.

In a third aspect, the invention is directed to a method to treat cancer in a subject, which comprises administering an effective amount of a topoisomerase I inhibitor coupled to a macromolecule through a linker that provides decoupling through a beta elimination mechanism in combination with an effective amount of a cell cycle checkpoint pathway inhibitor.

In the first aspect of the invention, the method may also include a procedure to diagnose the subject for the presence of the defect; and in embodiments where more than one agent (including the invention conjugates) is administered, the coadministration of more than one agent may be simultaneous or in sequence in either order of the agents. The difference in time of administration of coadministered agents may be as long as days. The agents may also optionally be administered in the same composition.

Combinations of the forgoing approaches are also included within the invention; thus, a subject inherently having a defect in DDR may be supplied the conjugated topoisomerase I inhibitor coupled to a macromolecule through a linker that provides decoupling through a beta elimination mechanism in combination with either an additional DDR inhibitor or a checkpoint pathway inhibitor or both. Independently, regardless of whether the subject exhibits an inherent defect in DDR, a combination of the topoisomerase I inhibitor conjugate of the invention with either or both an additional DDR inhibitor and a cell cycle checkpoint inhibitor is included within the scope of the invention. In addition, use of more than one DDR inhibitor and/or more than one cell cycle checkpoint inhibitor in combination with the topoisomerase I inhibitor conjugate is included in the scope of the invention.

### BRIEF DESCRIPTION OF THE DRAWINGS

FIG. 1 shows a schematic outline of the invention approach wherein single treatment with topoisomerase I inhibitor may be offset by various repair or cell cycle checkpoints as in panel A. When the subject has an inherent DDR defect, e.g., a mutation in the BRCA gene as in panel B, the effect of topoisomerase I inhibition is strengthened, and this is further strengthened by an inhibitor of DNA damage repair such as a PARP inhibitor as in panel C (PARP is poly ADP ribose polymerase).

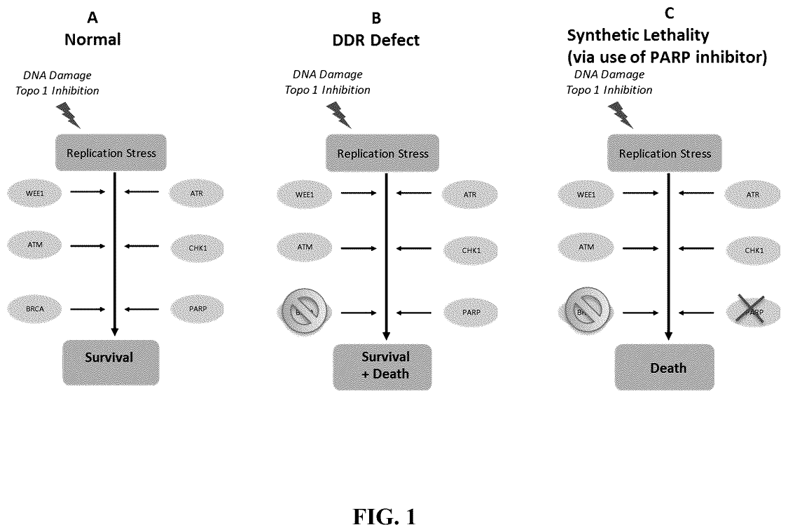

FIG. 2 shows the state of the art with regard to sensitivity to topoisomerase I inhibitors of various DDR defects, both with respect to germline in non-germline DDR associated with various genes.

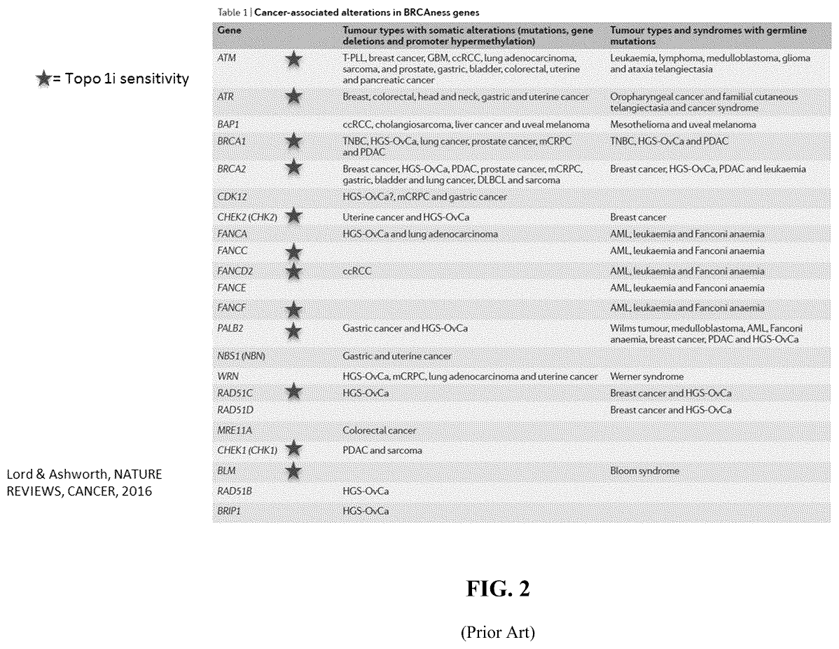

FIG. 3A-3C show the synergistic effect of an SN-38 conjugate of the invention with an inhibitor of PARP on tumor growth and on event free survival. Shown in FIG. 3A is a graph of tumor volume versus time. Shown is tumor volume for the vehicle control (●), and after single i.p. injection of 15 μmol/kg PLX038A (▪), daily oral gavage of 0.4 μmol/kg BMN673 (▴), or combination therapy of a single i.p. dose of 15 μmol/kg PLX038A with daily oral BMN673 starting on the same day (▾) or 4 days later (♦). When the vehicle control tumors reached ˜3,000 mm³, animals were treated with the combination, but BMN673 dosing was started immediately (◯). Data are plotted as mean tumor volumes±SEM. Groups had N=4 animals. FIG. 3B is a graph of relative tumor volume versus time. In FIG. 3B, daily TLZ was initiated 4 days after PLX038A dosing (♦). FIG. 3C shows event-free survival where an event is defined as a 4-fold increase over tumor size on day 0.

FIG. 4A-4C show the impact of BRCA 1 or BRCA 2 deficiency on effectiveness of the SN-38 conjugate in treating tumors in mice. Shown in FIG. 4A is a graph of relative tumor volume versus time in MX-1 xenografts. Shown is relative tumor volume for the vehicle control (●), and after single i.p. injections of 137 μmol/kg CPT-11 (▪), or 4- (♦), 40- (▾) or 120 μmol/kg PLX038A (▴). When the vehicle control tumors reached 2,000 mm³, animals were treated with 120 μmol/kg PLX038A (◯). Also shown are graphs of relative tumor volume versus time in CAPAN-1 BRCA2^{-/-} (FIG. 4B) and BRCA2^{+/+} (FIG. 4C) xenografts. Shown are the relative tumor volumes for the vehicle control (●) and after single i.p. injections of 137 μmol/kg CPT-11 (▪), or 15- (♦), 40- (▾) or 120 μmol/kg PLX038A (▴).

### MODES OF CARRYING OUT THE INVENTION

The invention takes advantage of synergistic attacks on the DNA damage response that might be mounted in cancer cells to affect their successful replication. The topoisomerase I inhibitor conjugates that cause DNA damage may be combined with either inhibitors of DDR or other inhibitors that interfere with DNA damage repair or replication. The DDR is an extremely complex process involving various mechanisms of fixing DNA to correct errors that occur either through mutation or through errors in the replication process itself. Part of this response is also a control mechanism involving cell cycle checkpoints that ensures that DNA is properly repaired or replicated before the cell divides or alternatively to effect apoptosis so that error-ridden DNA is not transmitted to daughter cells. The present invention employs a combination of a particular DDR inhibitor—a topoisomerase I inhibitor with other obstacles to successful replication including other inhibitors of DDR and inhibitors of cell cycle checkpoint pathways including instances wherein the cancer cells themselves are defective in their ability to respond to DNA damage.

The invention utilizes a conjugate of a topoisomerase I inhibitor coupled to a macromolecule through a linker that provides decoupling through a beta elimination mechanism. Suitable topoisomerase I inhibitors are typically camptothecin and analogs, including irinotecan, otherwise known as CPT-11, and its active metabolite, SN-38, as well as topotecan, 9-amino-camptothecin, and water soluble analogs, such as GI 147211 and GI 149893.

In some embodiments, the macromolecule is a linear or branched or multi-armed, polyethylene glycol.

Particularly preferred is a conjugate of formula (I)

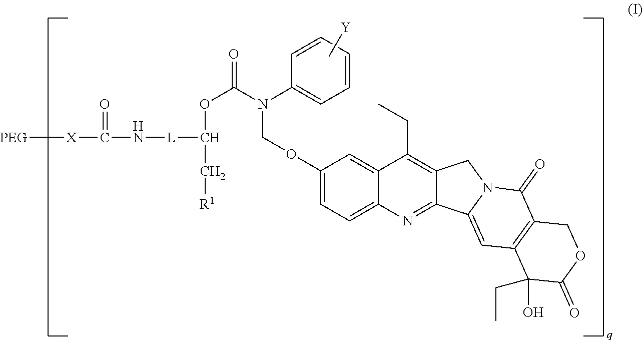

wherein
PEG is linear or branched and, when q is 2-8, multi-armed, polyethylene glycol;
X is (CH₂)ₘ, wherein m=1-6;
L is (CH₂CH₂O)ₚ(CH₂)ᵣ, wherein r=1-10 and p=0-10;
R¹ is CN or SO₂NR²₂, wherein each R² is independently alkyl, aryl, heteroaryl, alkylalkenyl, alkylaryl, or alkylheteroaryl, or two R² taken together can form a ring;
Y is COR³ or SO₂R³, wherein R³═OH, alkoxy, or NR⁴₂, wherein each R⁴ is independently alkyl, substituted alkyl, or two R⁴ taken together can form a ring; and
q is 1-8.

In particular, this conjugate may have a PEG of average molecular weight 30,000-50,000 Da, and/or wherein q=4, and/or wherein R¹═CN or SO₂NR²₂ wherein each R² is alkyl.

The conjugate may be of the formula:

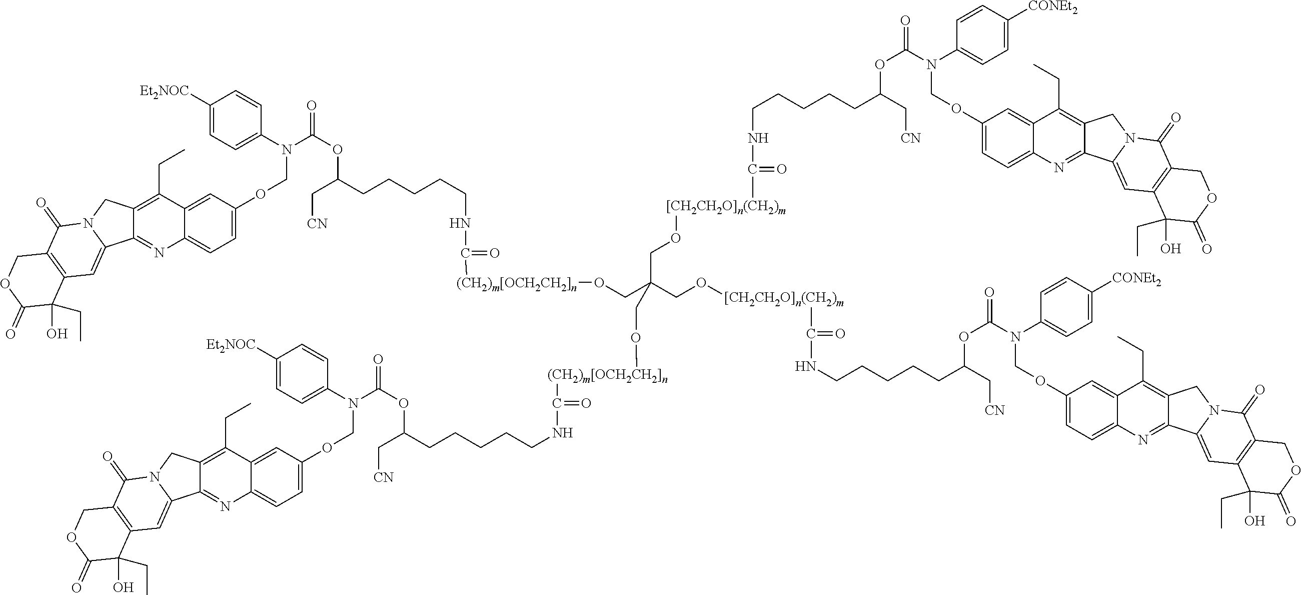

wherein m=1-6 and n is 200-250.

In particular, the conjugate may be PLX038, which is of the above formula where m is 1 and n is approximately 225.

The conjugates useful in the invention are generally provided in standard pharmaceutical formulations in combination with one or more pharmaceutically acceptable excipients, in some cases wherein the pH is between 4.0 and 6.0. Standard formulations can be found, for example, in Remington Pharmaceutical Sciences, Latest Edition, Mack Publishing Company, Easton, Pennsylvania.

The invention is based on the favorable properties of a conjugate that has suitable pharmacokinetics for combination with either endogenous DDR defects or with coadministered compounds that are cell cycle checkpoint inhibitors or DDR inhibitors.

In some embodiments, the conjugates, when administered to subjects provide a continuous low dose exposure to the topoisomerase I inhibitor wherein the concentration of the free inhibitor can be maintained between 15 and 5 nM between once or twice weekly administrations or over a protocol of administration, for example, of once every two weeks. In any case, the conjugates provide consistent low dose exposure to the active drug.

As to the identity of the coadministered DDR inhibitors and/or cell cycle checkpoint inhibitors, many are known in the art as set forth, for example, in the Background Art discussion above.

Cell cycle checkpoints include G1-S, S, and G2/M. Any of these can be targeted in combination with the topoisomerase I inhibitor conjugate, and/or in combination with additional agents that target components needed for successful checkpoint transition. This may be also against a background of an endogenous defect in cell cycle checkpoint control.

Suitable cell cycle checkpoint targets include checkpoint kinase 1 or 2 (CHK1 or CHK2), ataxia telangiectasia mutated (ATM) kinase, ataxia telangiectasia and Rad3 related (ATR) kinase, Wee1 kinase and p53. An extensive list of inhibitors of these targets is found in WO2012/074754.

Suitable DDR inhibitors include those that target homologous recombination (HR), e.g. poly(ADP-ribose) polymerase (PARP) inhibitors and/or other DDR pathways, including an HEJ, HR, alt-NHEJ/MMEJ, SSA, ICL, SSB, BER, TLS, NER and MMR. A large number of agents are in development for addressing these targets, and a number of agents known to do so are now used in the clinic.

All documents cited are incorporated herein by reference in their entirety.

The following example is intended to illustrate, but not limit the invention.

### Example 1

### Synergistic Effect of PLX038A and PARP Inhibitor Talazoparib (Designated BMN673 or TLZ)

Preparation of murine MX-1 xenografts: The MX-1 cell line was obtained from Charles River Labs (Frederick, Maryland).¹ Cells were cultured in RPMI-1640, 10% FBS and 1% 2 mM L-glutamine at 37° C. in 95% air/5% CO₂ atmosphere. ¹Ovejera A A et al. Chemotherapy of human tumor xenografts in genetically athymic mice. Ann Clin Lab Sci 8: 50-6, 1978.

Female NCr nude mice (N CrTac:NCr-Foxn1^{nu}; ˜6-7 weeks old) from Taconic Bioscience (Cambridge City, Indiana) were housed at the UCSF Preclinical Therapeutics Core vivarium (San Francisco, California). All animal studies were carried out in accordance with UCSF Institutional Animal Care and Use Committee. Tumor xenografts were established by subcutaneous injection with MX-1 tumor cells (2×10⁶ cells in 100 μl of serum free medium mixed 1:1 with Matrigel) into the right flank of female NCr nude mice. When tumor xenografts reached 1000-1500 mm³ in donor mice, they were resected, cut into even-size fragments (˜2.5×2.5×2.5 mm in size), embedded in Matrigel and re-implanted via subcutaneous trocar implantation in receiver mice.² ²Morton C L, Houghton P J. Establishment of human tumor xenografts in immunodeficient mice. Nat Protoc. 2007; 2(2):247-50.

Dosing and tumor volume measurements: Solutions of PLX038A (1.02 mM SN38; 0.26 mM PLX038A conjugate) were prepared in pH 5 isotonic acetate and sterile filtered (0.2 μm) before use. Solutions of BMN673 (52 μM) were prepared in 10% dimethylacetamide/5% Solutol HS15/85% 1×PBS and were sterile filtered (0.2 μm) before use.

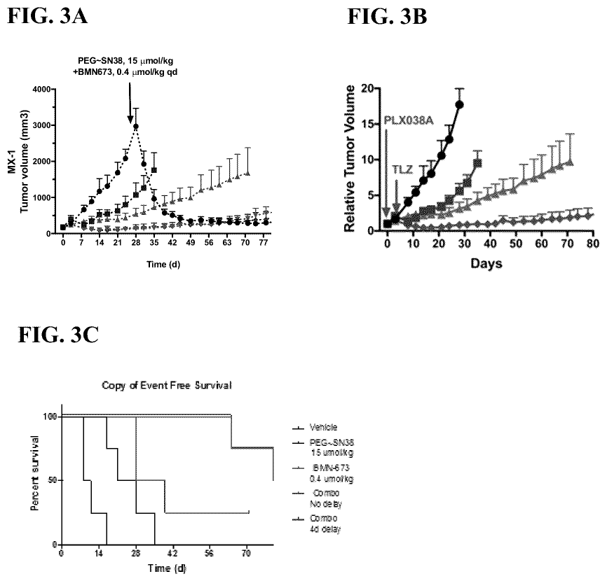

Groups (N=4-5/group) were dosed when the group average reached 100-200 mm³ in size. Mice received vehicle, a single dose of PLX038A (14.7 mL/kg i.p., 15 μmol/kg), daily doses of BMN673 (7.72 mL/kg p.o., 0.4 μmol/kg), or a combination of PLX038A and BMN673 at the same doses. For groups receiving the combination, daily BMN673 dosing began on the same day (FIG. 3A) or after a 4-day delay (FIG. 3B) after dosing PLX038A. Tumor volumes (caliper measurement: 0.5×(length×width²)) and body weights were measured twice weekly. When vehicle control tumors reached ˜3000 mm³ in size, mice were treated with the combination of a single dose of PLX038A (15 μmol/kg) and daily BMN673 (0.4 μmol/kg) combination with no delay between dosing (FIG. 3A).

As shown in FIGS. 3A and 3B, administration of PLX038A to mice bearing MX-1 tumors at 15 μmol/kg in combination with daily doses of Talazoparib at 0.4 μmol/kg provides a synergistic effect as compared to either of these drugs alone. This was true whether daily dosage with TLZ began at the same time as PLX038A or 4 days later. A single combination administered to control immediately reduced tumor volume (FIG. 3A).

As shown in FIG. 3C, event-free survival was enhanced synergistically with the combination vs PLX038A and TLZ individually.

### Example 2

### Synergy of PLX038A and Tumor Cell Defect

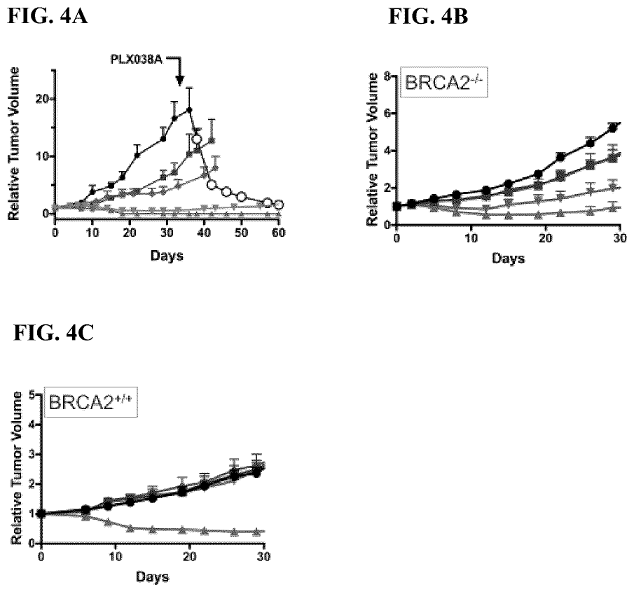

MX-1 cells are BRCA 1 deficient and CAPAN-1 cells are supplied as either BRCA 2 deficient (−/−) or not deficient (+/+). The general protocol of Example 1 was followed with mice bearing tumors of these cell lines. For mice with MX-1 tumors, dosages were single i.p. injections of 137 μg/kg of irinotecan or 4, 40 or 120 μg/kg of PLX038A. For mice with CAPAN-1 xenografts, dosages were single i.p. injections of 137 μg/kg irinotecan or 15, 40 or 120 μg/kg of PLX038A. FIG. 4A-4C show the results of these dosages on tumor volumes, which were measured twice weekly.

As shown in FIG. 4A, all dosages of PLX038A were more effective than irinotecan in reducing tumor volume, with 40 or 120 μg/kg essentially stopping tumor growth. Also shown is the dramatic result of a single dose of 120 μg/kg PLX038A administered when the control tumors reached 2000 mm³.

A comparison of FIGS. 4B and 4C shows the effect of BRCA 2 deficiency on the effectiveness of treatment with irinotecan or PLX038A—only the very highest dose of PLX038A was comparably effective for both deficient and non-deficient cells. The effectiveness of all other dosage levels was enhanced in the BRCA 2 deficient cells.

1. A method to treat cancer in a subject in need of such treatment, said subject having been diagnosed as having one or more defects in DNA damage response (DDR), which method comprises administering to said subject an effective amount of a conjugate of topoisomerase I inhibitor coupled to a macromolecule through a linker that provides decoupling through a beta elimination mechanism.

2. The method of claim 1 which includes administering to said subject an effective amount of an inhibitor of DDR in combination with said conjugate.

3. The method of claim 1 which includes administering to said subject an effective amount of a cell cycle checkpoint inhibitor in combination with said conjugate.

4. The method of claim 1 which further comprises diagnosing said subject for the presence or absence of said defect.

5. The method of claim 1 wherein the topoisomerase I inhibitor is SN-38.

6. The method of claim 5 wherein the macromolecule is polyethylene glycol (PEG).

7. The method of claim 5 wherein the conjugate is PLX038.

8. The method of claim 1 wherein the defect in DDR in the subject is a defect in homologous recombination repair (HRR) or in single strand break repair or the defect is in a tumor suppressor gene.

9. The method of claim 2 wherein the inhibitor of DDR is an inhibitor of HRR repair or of single strand break repair.

10. The method of claim 3 wherein the cell cycle checkpoint inhibitor is an inhibitor of CHK1, CHK2 or Wee1 kinase.

11. A method to treat cancer in a subject in need of such treatment, which method comprises administering to said subject an effective amount of a conjugate of topoisomerase I inhibitor coupled to a macromolecule through a linker that provides decoupling through a beta elimination mechanism in combination with an effective amount of an inhibitor of DDR.

12. The method of claim 11 wherein the inhibitor is an inhibitor of HRR repair or single strand break repair.

13. A method to treat cancer in a subject in need of such treatment, which method comprises administering to said subject an effective amount of a conjugate of topoisomerase I inhibitor coupled to a macromolecule through a linker that provides decoupling through a beta elimination mechanism in combination with an effective amount of a cell cycle checkpoint inhibitor.

14. The method of claim 13 wherein the cell cycle checkpoint inhibitor is an inhibitor of CHK1, CHK2 or Wee1 kinase.

15. The method of claim 11 wherein the topoisomerase I inhibitor is SN-38.

16. The method of claim 15 wherein the macromolecule is polyethylene glycol (PEG).

17. The method of claim 15 wherein the conjugate is PLX038.
